(19) United States
(12) Patent Application Publication
(10) Pub. No.: US 2023/0121179 A1
(43) Pub. Date: Apr. 20, 2023

(54) SKIN BARRIER FUNCTION ENHANCER

(71) Applicant: Shiseido Company, Ltd., Chiyoda-ku, Tokyo (JP)

(72) Inventors: Kazuyuki MIYAZAWA, Tokyo (JP); Renaud GILLET, Tokyo (JP); Bianca MCCARTHY, Tokyo (JP); Tetsuya KANEMARU, Tokyo (JP)

(73) Assignee: Shiseido Company, Ltd., Chuo-ku, Tokyo (JP)

(21) Appl. No.: 17/796,578

(22) PCT Filed: Jan. 29, 2021
(86) PCT No.: PCT/JP2021/003403

§ 371 Date: Jul. 29, 2022

(30) Foreign Application Priority Data
Jan. 31, 2020 (JP) 2020-015641

(51) Int. Cl. A61K 8/24; A61K 8/67; A61K 8/9789; A61Q 17/04

(52) U.S. Cl. CPC A61K 8/24; A61K 8/67; A61K 8/9789; A61Q 17/04

(57) ABSTRACT

An agent for enhancing skin barrier function was provided with.

An agent for enhancing skin barrier function comprising a wavelength conversion substance as an active ingredient; a composition and a product comprising the agent for enhancing skin barrier function; and a method for enhancing skin barrier function using thereof. The present invention can exhibit a desirable effect on skin by effectively making use of ultraviolet light to enhance skin barrier function.

### FIELD

The present invention relates to an agent for enhancing skin barrier function comprising a wavelength conversion substance, to a composition and product comprising the agent for enhancing skin barrier function , and to a method for enhancing skin barrier function in skin using the same.

### BACKGROUND

The harm to skin caused by ultraviolet light includes adverse effects such as skin cancer, photoaging, skin spots, wrinkles and inflammation, which are also undesirable from the viewpoint of health and beauty.

Many measures are therefore being taken to protect the skin from ultraviolet light. Such measures include the use of sunscreens, the implementation of indoor spaces for avoidance of sunlight, and the use of head coverings and clothing treated to block UV rays and films designed to block UV rays.

### Citation List

### Patent Literature

PTL 1] Japanese Patent Publication No. 6424656
PTL 2] Japanese Patent Publication No. 6361416
PTL 3] International Patent Publication No. 2018/004006
PTL 4] Japanese Unexamined Patent Publication No. 2018-131422
PTL 5] Japanese Unexamined Patent Publication HEI No. 5-117127
PTL 6] Japanese Patent Publication No. 4048420
PTL 7] Japanese Patent Publication No. 4677250
PTL 8] Japanese Patent Publication No. 3303942
PTL 9] Japanese Unexamined Patent Publication No. 2017-88719
PTL 10] International Patent Publication No. 2018/117117

### SUMMARY

### Technical Problem

It is an object of the present invention to provide a novel agent for enhancing skin barrier function that utilizes conversion of wavelength of ultraviolet light.

### Solution to Problem

The present inventors have conducted active research with the aim of allowing ultraviolet light to be effectively utilized on skin. As a result, an agent for enhancing skin barrier function has been devised by finding that the expression of barrier function related proteins are enhanced by irradiating ultraviolet light to skin cells through wavelength conversion substance that converts ultraviolet light wavelengths.

The present application provides the present invention with the aspects set forth below.
(1) An agent for enhancing skin barrier function comprising a wavelength conversion substance as an active ingredient, wherein the wavelength conversion substance converts the wavelength of ultraviolet light contained in incident light to emit emission light having a wavelength longer than the wavelength of the ultraviolet light.
(2) The agent for enhancing skin barrier function according to (1), wherein the ultraviolet light has a peak wavelength between 200 nm and 400 nm.
(3) The agent for enhancing skin barrier function according to (1) or (2), wherein the emission light has a peak wavelength between 450 nm and 700 nm.
(4) The agent for enhancing skin barrier function according to any one of (1) to (3), wherein the wavelength conversion substance comprises one or more phycobiliproteins selected from among allophycocyanin, C-phycocyanin, R-phycocyanin, phycoerythrocyanin, B-phycoerythrin, b-phycoerythrin, C-phycoerythrin and R-phycoerythrin; one or more inorganic phosphors selected from among zinc oxide phosphors, magnesium titanate phosphors and calcium phosphate phosphors; one or more components selected from among vitamin A, β-carotene, vitamin K, vitamin B1, vitamin B2, vitamin B6, vitamin B12, folic acid, niacin, lycopene, gardenia, safflower, turmeric, cochineal, perilla, red cabbage, flavonoids, carotenoids, quinoids, porphyrins, anthocyanins, and polyphenols; and/or one or more pigments selected from among Red No. 401, Red No. 227, Red No. 504, Red No. 218, Orange No. 205P, Yellow No. 4, Yellow No. 5, Green No. 201, Pyranin Conch, Blue No. 1, 2,4-diaminophenoxyethanol hydrochloride, Arizulin Purple SS, Violet No. 401, Black No. 401, Helindone Pink, Yellow No. 401, Bentizine Yellow G, Blue No. 404, Red No. 104, and meta-aminophenol,.
(5) The agent for enhancing skin barrier function according to (4), wherein the wavelength conversion substance comprises one or more phycobiliproteins selected from among allophycocyanin, C-phycocyanin, R-phycocyanin, phycoerythrocyanin, B-phycoerythrin, b-phycoerythrin, C-phycoerythrin and R-phycoerythrin; one or more inorganic phosphors selected from among zinc oxide phosphors, magnesium titanate phosphors and calcium phosphate phosphors; and/or one or more B vitamins selected from among vitamin B1, vitamin B2, vitamin B6 and vitamin B12.
(6) A composition comprising the agent for enhancing skin barrier function according to any one of (1) to (5).
(7) The composition according to (6), wherein the composition is a skin external composition for enhancing skin barrier function by exposing skin to light containing ultraviolet light.
(8) A cosmetic method for enhancing skin barrier function of a subject, comprising: applying the composition according to (6) or (7) to skin of a subject, and exposing the composition-applied skin with light containing ultraviolet light.
(9) A product comprising the agent for enhancing skin barrier function according to any one of (1) to (5).
(10) The product according to (9), wherein the product is for enhancing skin barrier function in skin by exposing the skin to light passing through the product, which contains ultraviolet light.
(11) A cosmetic method for enhancing skin barrier function in skin of a subject, comprising: bringing light containing ultraviolet light through the product according to (9) or (10), and exposing the skin of the subject to light passing through the product.

### Advantageous Effects of Invention

The present invention can enhance skin barrier function in skin cells by effectively making use of ultraviolet light, and is based on finding that enhanced skin barrier function results in a desirable effect on skin. The invention provides novel uses of the aforementioned compounds that have conventionally been used primarily as dyes, pigments, ultraviolet scattering agents, ultraviolet absorbers, nutrients and antioxidants. The invention also helps to improve quality of life by providing a more positive feeling for persons who have attempted to avoid ultraviolet light as much as possible for beauty or health reasons when outdoors.

### BRIEF DESCRIPTION OF DRAWINGS

FIG. 1 is a schematic diagram illustrating Experiment 1.

FIG. 2 shows changes in the expression levels of skin barrier function-related genes in cultured cells when UV is irradiated along with using Lumate G as a wavelength-conversion substance in Experiment 3 (A:SMPD1, B: FLG, C: INV, D: CDSN, E: TGase1). The vertical axis is the relative amount 2^{-ΔΔCt} for SMPD1 and TGase1, and the average of ΔCt values for FLG,INV, and CDSN.

FIG. 2 shows changes in the expression levels of skin barrier function-related genes in cultured cells when UV is irradiated along with using Lumate G as a wavelength-conversion substance in Experiment. 3 (A:SMPD1, B: FLG, C: INV, D: CDSN, E: TGase1). The vertical axis is the relative amount 2^{-ΔΔCt} for SMPD1 and TGase1 and the average of ΔCt values for FLG,INV, and CDSN.

FIG. 2 shows changes in the expression levels of skin barrier function-related genes in cultured cells when UV is irradiated along with using Lumate G as a wavelength-convertsion substance in Experiment. 3 (A:SMPD1, B: FLG, C: INV, D: CDSN, E: TGase1). The vertical axis is the relative amount 2^{-ΔΔCt} for SMPD1 and TGase1 and the average of ΔCt values for FLG, INV, and CDSN.

### DESCRIPTION OF EMBODIMENTS

The agent for enhancing skin barrier function of the invention comprises a wavelength conversion substance as an active ingredient. A wavelength conversion substance is a substance that converts the wavelength of ultraviolet light contained in incident light and emits emission light having a wavelength longer than the wavelength of the ultraviolet light.

The ultraviolet light may include UVA, UVB and UVC. According to one embodiment, the ultraviolet light is light with a peak wavelength of 200 nm to 400 nm. The ultraviolet light may also be included in incident light such as sunlight, for example. Alternatively, the incident light may be ultraviolet light, and artificially generated ultraviolet light may be used. Ultraviolet light can have various effects on the skin. As an example, ultraviolet light is known to cause sunburns, such as sunburn and suntan, and to cause DNA damage in cells. Cellular activity is altered in ultraviolet-irradiated cells, which causes altered gene expression. As an example, UV irradiation reduces gene expression of skin barrier function-related proteins.

The emission light emitted by the wavelength conversion substance has a longer wavelength than ultraviolet light, with a peak wavelength of preferably 450 nm to 700 nm. The emission light may have one or more peaks at 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm or 700 nm, or in any range within these values, though without being restrictive, or it may be red light, orange light, green light or blue light. According to one embodiment, the wavelength conversion substance has its main wavelength at 450 nm to 700 nm, for example 450 - 700 nm, for light emitted upon excitation with excitation light of 200 nm to 400 nm.

Examples of wavelength conversion substances include the following components: phycobiliproteins such as allophycocyanin, C-phycocyanin, R-phycocyanin, phycoerythrocyanin, B-phycoerythrin, b-phycoerythrin, C-phycoerythrin and R-phycoerythrin; natural or synthetic components such as vitamin A, β-carotene, vitamin K, vitamin B1, vitamin B2, vitamin B6, vitamin B12, folic acid, niacin, lycopene, gardenia, safflower, turmeric, cochineal, perilla, red cabbage, flavonoids, carotenoids, quinoids, porphyrins, anthocyanins, polyphenols; dyes such as Red No. 401, Red No. 227, Red No. 504, Red No. 218, Orange No. 205 P, Yellow No. 4, Yellow No. 5, Green No. 201, Pyranin Conch, Blue No. 1, 2,4-diaminophenoxyethanol hydrochloride, Arizulin Purple SS, Violet No. 401, Black No. 401, Helindone Pink, Yellow No. 401, Bentizine Yellow G, Blue No. 404, Red No. 104, meta-aminophenol; and phosphors obtained by fluorescent-doping of inorganic compounds, for example, blue phosphors comprising the amorphous silica particles mentioned in Japanese Patent No. 6424656, cerium and phosphorus and/or magnesium, and red phosphors comprising compounds obtained by europium activation of mixed crystals consisting of the alkaline earth metal sulfides described in Japanese Patent No. 6361416 combined with gallium compounds, the zinc oxide phosphors mentioned in International Patent Publication No. 2018/004006, the zinc oxide phosphors mentioned in Japanese Unexamined Patent Publication No. 2018-131422, and the inorganic phosphors mentioned in Japanese Unexamined Patent Publication HEI No. 5-117127. According to one embodiment, the inorganic phosphor is one or more phosphors selected from among phosphors obtained by doping zinc oxides represented by ZnO: Zn, Zn_{1+z}, ZnO₁₋ₓ with the sulfur-containing compounds mentioned in International Patent Publication No. 2018/004006, including sulfides and/or sulfates such as zinc sulfide or zinc sulfate, magnesium titanate phosphors obtained by doping magnesium titanates such as MgTiO₃ or Mg₂TiO₄ with manganese, and calcium phosphate phosphors obtained by doping calcium phosphates such as Ca(H₂PO₄)₂, CaHPO₄ or Ca₃(PO₄)₂ with cerium.

The wavelength conversion substance may be obtained by extraction from a natural source such as an animal, plant or algae, or it may be obtained by an artificial method such as chemical synthesis. For example, phycobiliproteins can be prepared by extraction from algae, including blue-green algae such as spirulina (Spirulina platensis) or red algae such as porphyridiophylla (Porphyridium purpureum), by the method described in Japanese Patent No. 4048420, Japanese Patent No. 4677250 or Japanese Patent No. 3303942, for example. Zinc oxide phosphors can be produced by the method described in International Patent Publication No. 2018/004006, Japanese Unexamined Patent Publication No. 2018-131422 or Japanese Unexamined Patent Publication HEI No. 5-117127, for example. Magnesium titanate phosphors can be produced by the method described in Japanese Unexamined Patent Publication No. 2017-88719. Calcium phosphate phosphors can be produced by the method described in International Patent Publication No. 2018/117117.

So long as the wavelength conversion effect of the invention is not impaired, these wavelength conversion substances may be composed of, or may include, the components mentioned above, and they may be single components alone or combinations of more than one of the components. For example, the aforementioned phycobiliproteins or inorganic material phosphors may be mixed with other wavelength conversion substances such as B vitamins (vitamin B1, vitamin B2, vitamin B6 or vitamin B12) to exhibit synergistic effects. These components are merely examples, however, and any other substances that exhibit the wavelength conversion effect of the invention may be used.

The wavelength conversion substance content in the agent for enhancing skin barrier function, composition or product of the invention is not particularly restricted so long as the wavelength conversion effect of the invention is not impaired, and it may be appropriately determined for the type of wavelength conversion substance and the purpose of use of the agent for enhancing skin barrier function or composition. It may be any content in the range of 0.01 to 99.99 wt% or 0.1% to 999 wt%, for example.

When ultraviolet light is irradiated to the skin barrier function enhancing agent of the present invention, it emits emission light. Emission light can enhance the expression of skin barrier function-related proteins in skin cells, thereby exerting an effect for enhancing skin barrier function. Enhanced skin barrier function may be an effect that restores skin barrier function reduced by ultraviolet light, or may be an effect that enhances skin barrier function reduced by causes other than ultraviolet light. An agent for enhancing skin barrier function can also be referred to as an agent for improving skin barrier function. The agent for enhancing skin barrier function of the present invention can be used for any subject, but may be applied to a subject exposed to ultraviolet rays outside, or to a subject having a reduced skin barrier function.

Skin barrier function-related proteins are directed to proteins that can enhance the amount of any substance that enhances skin barrier function in the epidermis, or can promote the formation of structures that enhance skin barrier function. As skin barrier function-related proteins, they may be proteins that constitute the structure of the stratum corneum, or may be proteins that are involved in the production of components or the formation of structures, which are capable of enhancing skin barrier function. Skin barrier function-related proteins include at least one selected from comeodesmosine (CDSN), sphingomyelin phosphodiesterase (SMPD1), filaggrin (FLGs), involucrin (INVs), loricrin (LORs), transglutaminase 1 (TGase1), and caspase 14 (CASP14). The agent for enhancing skin barrier function of the present invention can absorb ultraviolet rays and promote the expression of the skin barrier function-related protein by emitting emission light. Accordingly, the agent for enhancing skin barrier function of the present invention may also be referred to as an expression promoting agent of a skin barrier function related protein.

Comeodesmosine (CDSN) is a cell-adhesion protein present in corneodesmosomes that adhere between keratinocytes. It has been reported that the mutation has been added to the gene of corneodesmosin in the PSD (peeling skin disease) patient, and that stratum corneum is peeled off by the malfunction of comeodesmosin. Corneodesmosine contributes to skin barrier function because it adheres between keratinocytes.

Sphingomyelin phosphodiesterase 1 (SMPD1) is an enzyme that metabolizes sphingomyelin, a type of sphingolipid. Degradation of sphingomyelin results in the formation of phosphorylcholine and ceramide. During differentiation from the granular layer to the stratum corneum, sphingomyelin is degraded by the action of SMPD1 to form ceramide. Generated ceramides contribute to skin barrier function as intercellular lipids.

Filaggrin (FLG) is a protein produced in granule cells of the epidermis. It is synthesized as a precursor profilaggrin, and during the formation of the stratum corneum, it undergoes dephosphorylation and hydrolysis to generate filaggrin. The generated filaggrin binds to keratin in the cytoplasm and aggregates keratin. Filaggrin is subsequently degraded to amino acids by degrading enzymes such as caspase 14 and functions as a natural moisturizing factor. The produced natural moisturizing factors contribute to skin barrier function.

Involculin (INV) is a protein generated in spiny cells. Involculin and loricrin, which is generated in granule cells, are major components of the peripheral zone. Involculin and loricrin are cross-linked by transglutaminase during keratinization to form insoluble structures that line the plasma membrane of keratinocites as a peripheral zone. Thus, the crosslinked involucrin imparts strength to the stratum corneum and also contributes to the skin barrier function.

Loricrin (LOR) is a protein generated in granule cells. Loricrin and involucrin are major components of the peripheral zone. Loricrin and involucrin are cross-linked by transglutaminase during keratinization to form insoluble structures that line the plasma membrane of corneocytes as a peripheral zone. Thus crosslinked loricrin provides strength to the stratum corneum and also contributes to skin barrier function.

Transglutaminase 1 (TGase1) is a protein-crosslinking enzyme. TGase 1 forms Cross-links between glutamine residues in proteins and lysine residues of heterologous or homologous proteins. The activity of transglutaminase 1 is regulated by calcium levels. During the process of differentiating granule cells into the corneocyte, cell death is occured, and the influx of calcium into the cytoplasm activities transglutaminase 1 in the corneocyte. Activated transglutaminase 1 cross-links loricrin and involucrin, thereby forming the peripheral zone. The peripheral zone lines the plasma membrane of corneocytes. This provides strength to the stratum corneum and also contributes to skin barrier function.

Caspase-14 (CASP14) is a cysteine protease. It is expressed as pro-caspase 14 in granule cells from spiny cells and is activated in the stratum corneum. Active caspase 14 degrades part of filaggrin. The action of additional enzymes on degraded filaggrin produces natural moisturizing factors (NMFs), which contribute to skin barrier function.

Any form of administration may be used for the agent for enhancing skin barrier function and composition of the invention, but an external preparation for skin will often be preferred, such as a drug, quasi drug or cosmetic, for enhancing skin barrier function by exposing the skin to light containing ultraviolet light. When the agent for enhancing skin barrier function or composition of the invention is to be used as an external preparation for skin, the dosage form, coating method and number of doses may be determined as desired. For example, it may be applied onto skin in the form of cosmetic water or a spray, oil, cream, latex, gel, sunscreen or suntan lotion either periodically or irregularly, once or several times per day at morning, noon or evening, or before going out or engaging in outdoor activities, marine sports or skiing, for example, when exposure to sunlight is expected.

The agent for enhancing skin barrier function and composition of the invention may also be used in combination with an additive such as an excipient, preservative, thickener, binder, disintegrator, dispersing agent, stabilizer, gelling agent, antioxidant, surfactant, preservative, oil, powder, water, alcohol, thickener, chelating agent, silicone, antioxidant, humectant, aromatic, drug component, antiseptic agent, pH adjustor or neutralizer, selected as necessary or desired. It may also be used in combination with other agent for enhancing skin barrier function to increase the effect of the invention.

The present invention further provides products such as sun visors, caps, clothing, gloves, screen films, window sprays or creams, window materials or wall materials, for example, that comprise the agent for enhancing skin barrier function of the invention and are intended to enhance skin barrier function by exposing the skin to light containing ultraviolet light. The usage of additives in the products of the invention and the forms of the products may also be as desired.

The present invention further provides a method for producing the agent for enhancing skin barrier function, composition or product of the invention. A method for enhancing skin barrier function in the skin of a subject is also provided, the method comprising application of the agent for enhancing skin barrier function or composition of the invention onto the skin of a subject and exposing the skin to light containing ultraviolet light after application of the agent for enhancing skin barrier function or composition; or passing light containing ultraviolet light through the product of the invention, and exposing the skin to the transmitted light; wherein the agent for enhancing skin barrier function, composition or product converts the wavelength of ultraviolet light in the incident light and emits emission light with a longer wavelength than the wavelength of the ultraviolet light, transmitting the ultraviolet light with a peak wavelength of preferably 200 nm to 400 nm as light with a peak wavelength of 450 nm to 700 nm, for example 500 nm to 700 nm. The method for enhancing skin barrier function in skin of an subject will often be for the purpose of beautifying, instead of treatment by a doctor or medical worker.. The invention further provides a cosmetic counseling method for supporting cosmetology, which includes providing a cosmetic method, an agent for enhancing skin barrier function, composition or product of the invention to a subject.

### EXAMPLES

The present invention will now be explained in greater detail by examples. However, the invention is in no way limited by the examples.

Experiment 1: Change in gene expression by applying wavelength conversion substances Experiment 1-1: Preparation of wavelength conversion substances Wavelength conversion substances were prepared in the following manner.
(1) C-phycocyanin C-phycocyanin is obtained from spirulina (Spirulina platensis) extract, the absorption spectrum having a peak wavelength at 350 nm and the emission spectrum having peak wavelengths at 640 nm and 700 nm.
(2) Riboflavin (vitamin B2) Riboflavin, also called vitamin B2, had a peak wavelength at 445 nm and an emission spectrum had a peak wavelength at 530 nm.
(3) Zinc oxide phosphor Lumate G by Sakai Chemical Industry Co., Ltd. was used. Lumate G is a zinc oxide phosphor obtained by doping ZnO with a sulfur-containing compound and then firing as described in International Patent Publication No. 2018/004006, the absorption spectrum having a peak wavelength at 365 nm and the emission spectrum having a peak wavelength at 510 nm.
(4) Magnesium titanate phosphor Lumate R by Sakai Chemical Industry Co., Ltd. was used. Lumate R is a magnesium titanate phosphor obtained by doping MgTiO₃ with manganese, the absorption spectrum having a peak wavelength at 365 nm and the emission spectrum having peak wavelengths in the range of 660 to 680 nm.
The wavelength conversion substances of (1) and (2) were dissolved in water to prepare solutions at concentrations of 1% and 5%.
The wavelength conversion substances of (3) and (4) were dispersed in alcohol to prepare 5% and 10% dispersions.

### Experiment 1-2: Preparation of Cell Samples

Cell samples were prepared in the following manner.
1. Human human skin keratinocytes (Normal Human Epidermal Keratinocytes, PromoCell) were used. A cell suspension (1 mL) stored with liquid nitrogen was placed in a hot water bath (37° C.) and thawed until small ice pellets remained, and then diluted with 9 mL of warm KGM medium.
2. The diluted suspension was gently mixed and transferred to a T75 flask, and incubated overnight at 37° C.
3. On the following day, the medium was exchanged with 10 mL of fresh medium.
4. The medium was periodically exchanged (once every 2 - 3 days), while continuing growth of the cells. During this time, a microscope was used to observe the cells and to confirm that the cells had proliferated with the proper form.
5. Once the cells reached approximately 80% confluence, they were subcultured.
6. Subculturing of the cells was carried out by rinsing and aspirating once in 10 mL of warm PBS.
7. 5 mL of warm trypsin was added to the T75 flask to cover the bottom of the flask with the trypsin solution, and the mixture was aspirated after standing for 1 minute at room temperature.
8. The flask was set in an oven at 37° C. for 5 minutes (maximum) for keratinocytes. A microscope was used to observe the cells, confirming that they were small and elliptical.
9. The sides of the T75 flask were then lightly tapped to free the cells. A microscope was used to observe the cells and confirm that they were freely moving.
10. The keratinocytes were resuspended in 5 mL of warm trypsin neutralizing solution, transferred to a sterilized 50 mL Falcon tube. The flask was further rinsed with 5 mL of warm FGM and added to the Falcon tube, to ensure transfer of all of the cells.
11.. The cells were centrifuged at 10,000 rpm for 5 minutes (4° C.), and the supernatant was carefully removed while avoiding disturbing the cell pellet.
12. The keratinocytes were resuspended in KGM at a concentration of 2 × 10⁴ cells/well (500 µL), and plated in collagen coated glass bottom 4-well chamber slide.
13. The medium was replaced every two or three days, and cells were proliferated until they reached 60 to 70% confluence (differing for the type of experiment).
14. At 24 hours before irradiation, the medium was changed to non-supplemented medium.

### Experiment 1-3: Ultraviolet Light Irradiation

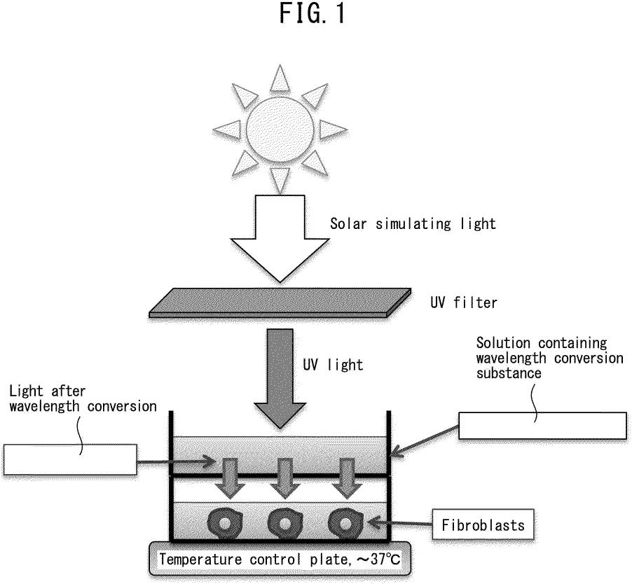

1. At least 30 minutes prior to irradiation, the power source of a solar simulator was activated to warm up the lamp. The solar simulator used was a UG11 filter. A UG11 filter is a filter that allows passage of UVB alone while cutting light of other wavelengths. The UV light passing through the UG11 filter had a peak wavelength of 300 nm to 385 nm.
2. The temperature control plate was turned on and set to 33° C.
3. The cells prepared in Experiment 1-2 were rinsed once with warm PBS.
4. A 0.5 mL portion of warmed Martinez solution (145 mM NaCl, 5.5 mM KC1, 1.2 mM MgCl₂·6H₂O, 1.2 mM NaH₂PO₄·2H₂O, 7.5 mM HEPES, 1 mM CaCl₂ and 10 mM D-glucose) was added to each well.
5. As shown in FIG. 1, the cell-containing wells were set on a plate, 0.4 ml of each solution containing the wavelength conversion substances (1) to (4) prepared in Experiment 1-1 was injected into the wells of a 24-well plate, the cell-containing wells were placed over it in a manner covering the wells, and UV light was irradiated into the cell solution through the wavelength conversion substance solution without allowing direct contact between the wavelength conversion substance solution and the cell solution.
6. The irradiation was carried out to a total radiation dose of 100 mJ/cm². As controls, there were prepared a sample of the cells directly irradiated with UV light without setting a wavelength conversion substance plate on the cell-containing wells, and a sample of the cells cultured in a dark environment without irradiation of UV light.
7. After irradiation, the Martinez solution was exchanged with warmed KGM (supplement-free) and the plate was returned to the 37° C. incubator, and incubated for 24 hours.

### Experiment 2: Microarray

Experiment 2-1: RNA extraction Cell samples incubated for 24 h after irradiating ultraviolet light in Experiments 1-3 were washed with 500 µl warm PBS, and PBS was completely aspirated. Qiagen RNeasy Mini Kit prep (Qiagen,74106) were used to extract RNA according to the product instructions.

### Experiment 2-2: Microarray

Microarray for Human gene-expression (SurePrint G3 Human GE Microarray 8x60K Ver. 3.0 (Agilent technology)) was used to perform the analysis of RNAs extracted in Experiment 2-1. The extracted RNA was subjected to labeling reactions, amplification reactions, purification, and quantitation of cRNA according to the protocol provided by Agilent Technology to prepare hybridization samples.. Microarrays were observed with AGILINT C MICROARRAY SCANNER to identify genes whose expression was significantly reduced or increased by the presence or absence of wavelength-converting material, and the results are shown below.

TABLE 1

| | | | | |
|---|---|---|---|---|
| Gene name | Lina Blue | Vitamin B2 | Lumate G | Lumate R |
| SMPD1 | O | O | X | X |
| FLG | O | O | O | O |
| INV | O | O | O | O |
| LOR | O | X | X | X |
| CDSN | O | O | O | X |
| TGase1 | O | O | X | X |
| CASP14 | O | O | X | O |

O represents the gene whose expression is significantly changed, and
X represents the gene whose expression is not changed.

### Experiment 3: RT-PCR

Experiment 3-1: RNA extraction
1. The cell samples incubated for 24 h after UV irradiation in Experiments 1-3 was subjected to RNA extraction by using RNeasy Kit (Qiagen) in accordance with the product instructions to extract RNA.
2. Concentration and A260/280 were recorded for each sample.

### Experiment 3-2: Reverse Transcription

1. SuperScript VILO cDNA Synthesis Kit (Thermo Fisher) was used in accordance with the product instructions, with 1 pg to 2.5 µg of RNA being added per container, and the PCR system was operated at a setting of at 25° C. for 10 min, at 42° C. for 60 min, at 85° C. for 5 min, and preservation at 4° C.

### Experiments 3-3: RT-PCR

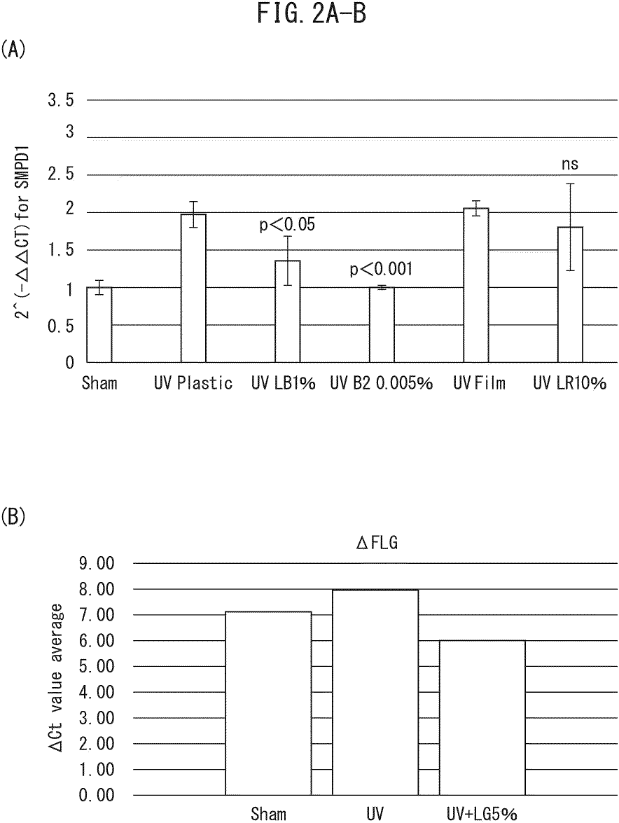

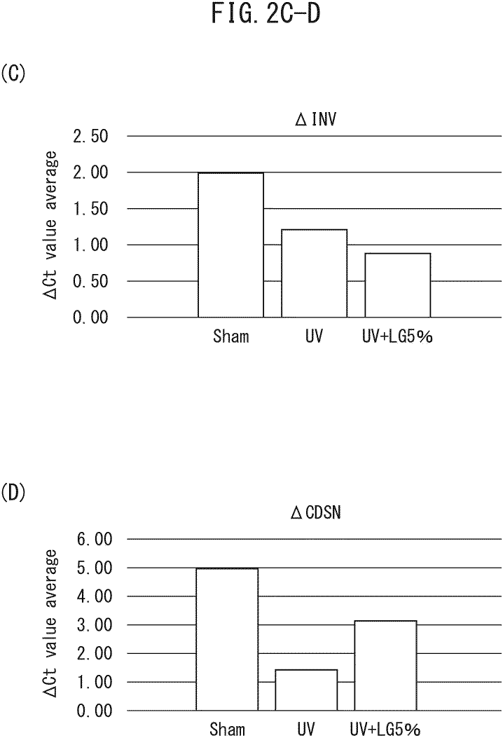

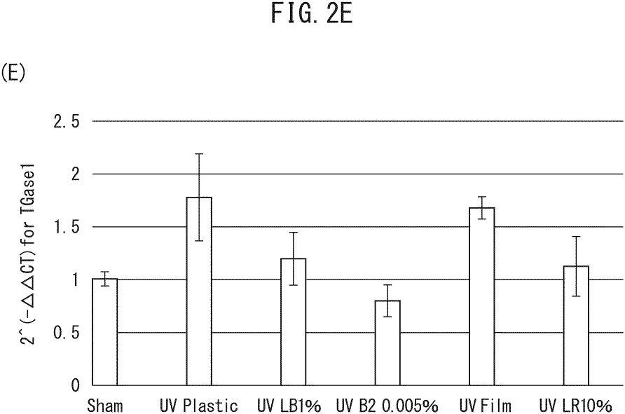

The reverse-transcripted sample was diluted 50-fold with RNase free water, and a further 5-fold dilution series was prepared. Then, the reaction system described below was prepared and measured by a real-time PCR instrument (Applied Biosystems). ΔCt was determined based on the Ct value for each gene and the Ct value of the internal standard, GAPDH in the test sample (FLGs, INVs, and CDSN). In addition, ΔΔCt was determined for Sham sample (UV-unirradiated sample) and calculated as relative amount 2^{-AACt} (SMPD1 and TGase1) (FIG. 2).

TABLE 2

| Reaction system | µl/well |
|---|---|
| unamplified diluted cDNA | 5 |
| Platinum Sybr green qPCR Super Mix-UDG | 12.5 |
| Primer mix (F/R) (5 µM) | 1 |
| ROX Reference Dye | 0. 5 |
| RNase free water | 6 |
| Total | 25 |

TABLE 3

| Primer name | Sequence | Sea ID No. |
|---|---|---|
| SMPD1 Forward | TGGCACCCAGTGCAACTACCTA | 1 |
| SMPD1 Reverse | AGAAGCTGCCAGTGCGGTATG | 2 |
| FLG Forward | GGCAAATCCTGAAGAATCC | 3 |
| FLG Reverse | TGCTTTCTGTGCTTGTGTCC | 4 |
| INV Forward | GATGTCCCAGCAACACACAC | 5 |
| INV Reverse | TGCTCACATTCTTGCTCAGG | 6 |
| CDSN Forward | CCCATCTCTGAGGGCAAATA | 7 |
| CDSN Reverse | CTAGAACTGCTGGGGACTCG | 8 |
| TGase1 Forward | GAGCGGAAGGCAGTAGAGACA | 9 |
| TGase1 Reverse | CCCGGGTTGGCATACACA | 10 |
| GAPDH Forward | GAAGGTGAAGGTCGGAGTC | 11 |
| GAPDH Reverse | GAAGATGGTGATGGGATTTC | 12 |

These results demonstrates that the expression of skin barrier function-related proteins was promoted by irradiating UV to wavelength-converting materials. Thereby, an effect of enhancing skin barrier function was exerted.

The embodiments of the invention described above are not intended to place limitations on the invention, and various modifications including cosmetics and drug compositions may be incorporated, which fall within the gist of the invention.

1. An agent for enhancing skin barrier function comprising a wavelength conversion substance as an active ingredient,
wherein the wavelength conversion substance converts the wavelength of ultraviolet light contained in incident light to emit emission light having a wavelength longer than the wavelength of the ultraviolet light.

2. The agent for enhancing skin barrier function according to claim 1, wherein the ultraviolet light has a peak wavelength between 200 nm and 400 nm.

3. The agent for enhancing skin barrier function according to claim 1 or 2, wherein the emission light has a peak wavelength between 450 nm and 700 nm.

4. The agent for enhancing skin barrier function according to any one of claims 1 to 3, wherein the wavelength conversion substance comprises one or more phycobiliproteins selected from among allophycocyanin, C-phycocyanin, R-phycocyanin, phycoerythrocyanin, B-phycoerythrin, b-phycoerythrin, C-phycoerythrin and R-phycoerythrin; one or more inorganic phosphors selected from among zinc oxide phosphors, magnesium titanate phosphors and calcium phosphate phosphors; one or more components selected from among vitamin A, β-carotene, vitamin K, vitamin B1, vitamin B2, vitamin B6, vitamin B12, folic acid, niacin, lycopene, gardenia, safflower, turmeric, cochineal, perilla, red cabbage, flavonoids, carotenoids, quinoids, porphyrins, anthocyanins, polyphenols; and/or one or more pigments selected from among Red No. 401, Red No. 227, Red No. 504, Red No. 218, Orange No. 205P, Yellow No. 4, Yellow No. 5, Green No. 201, Pyranin Conch, Blue No. 1, 2,4-diaminophenoxyethanol hydrochloride, Arizulin Purple SS, Violet No. 401, Black No. 401, Helindone Pink, Yellow No. 401, Bentizine Yellow G, Blue No. 404, Red No. 104, and meta-aminophenol.

5. The agent for enhancing skin barrier function cell activator according to claim 4, wherein the wavelength conversion substance comprises one or more phycobiliproteins selected from among allophycocyanin, C-phycocyanin, R-phycocyanin, phycoerythrocyanin, B-phycoerythrin, b-phycoerythrin, C-phycoerythrin and R-phycoerythrin; one or more inorganic phosphors selected from among zinc oxide phosphors, magnesium titanate phosphors and calcium phosphate phosphors; and/or one or more B vitamins selected from among vitamin B1, vitamin B2, vitamin B6 and vitamin B12.

6. A composition comprising the agent for enhancing skin barrier function according to any one of claims 1 to 5.

7. The composition according to claim 6, wherein the composition is a skin external composition for enhancing skin barrier function in skin by exposing skin to light containing ultraviolet light.

8. A cosmetic method for enhancing skin barrier function in skin of a subject, comprising:
applying the composition according to claim 6 or 7 to skin of a subject, and
exposing the composition-applied skin with light containing ultraviolet light.

9. A product comprising the agent for enhancing skin barrier function according to any one of claims 1 to 5.

10. The product according to claim 9, wherein the product is for enhancing skin barrier function in skin by exposing the skin to light passing through the product, which contains ultraviolet light.

11. A cosmetic method for enhancing skin barrier function in skin of a subject, comprising:
bringing light containing ultraviolet light through the product according to claim 9 or 10, and
exposing the skin of the subject to light passing through the product.


SEQUENCE LISTING (12 sequences)

SEQ ID NO: 1 - DNA, 22 bp, Artificial Sequence
  Primer
tggcacccagtgcaactaccta

SEQ ID NO: 2 - DNA, 21 bp, Artificial Sequence
  Primer
agaagctgccagtgcggtatg

SEQ ID NO: 3 - DNA, 19 bp, Artificial Sequence
  Primer
ggcaaatcctgaagaatcc

SEQ ID NO: 4 - DNA, 20 bp, Artificial Sequence
  Primer
tgctttctgtgcttgtgtcc

SEQ ID NO: 5 - DNA, 20 bp, Artificial Sequence
  Primer
gatgtcccagcaacacacac

SEQ ID NO: 6 - DNA, 20 bp, Artificial Sequence
  Primer
tgctcacattcttgctcagg

SEQ ID NO: 7 - DNA, 20 bp, Artificial Sequence
  Primer
cccatctctgagggcaaata

SEQ ID NO: 8 - DNA, 20 bp, Artificial Sequence
  Primer
ctagaactgctggggactcg

SEQ ID NO: 9 - DNA, 21 bp, Artificial Sequence
  Primer
gagcggaaggcagtagagaca

SEQ ID NO: 10 - DNA, 18 bp, Artificial Sequence
  Primer
ccccggttggcatacaca

SEQ ID NO: 11 - DNA, 19 bp, Artificial Sequence
  Primer
gaaggtgaaggtcggagtc

SEQ ID NO: 12 - DNA, 20 bp, Artificial Sequence
  Primer
gaagatggtgatgggatttc